(19) United States
(12) Patent Application Publication
(10) Pub. No.: US 2014/0030289 A1
(43) Pub. Date: Jan. 30, 2014

(54) COMPOSITIONS COMPRISING BETA-CARYOPHYLLENE AND METHODS OF UTILIZING THE SAME

(71) Applicants: Rivka Ofir, Arava (IL); Shimon Rachmilevich, Negev (IL); Eitan Amiel, Beer Sheva (IL); Nativ Dudai, Kfar Yehezkel (IL); Tattiana Rabinski, Be'er Sheva (IL); Shimshon Ben-Yehoshua, Kiryat Ono (IL)

(72) Inventors: Rivka Ofir, Arava (IL); Shimon Rachmilevich, Negev (IL); Eitan Amiel, Beer Sheva (IL); Nativ Dudai, Kfar Yehezkel (IL); Tattiana Rabinski, Be'er Sheva (IL); Shimshon Ben-Yehoshua, Kiryat Ono (IL)

(21) Appl. No.: 13/983,798

(22) PCT Filed: Feb. 6, 2012
(86) PCT No.: PCT/IL12/00065

§ 371 Date: Oct. 21, 2013

Related U.S. Application Data
(60) Provisional application No. 61/439,892, filed on Feb. 6, 2011.

(51) Int. Cl. A61K 31/015; C12N 5/09

(52) U.S. Cl. CPC A61K 31/015; C12N 5/0693
USPC 424/195.18; 435/375; 514/766; 424/725; 424/774; 424/779

(57) ABSTRACT

The invention provides compositions and methods for inducing apoptosis in a cancer cell or a cell transformed by Epstein-Barr virus (EBV). Specifically, the invention provides Beta-Caryophyllene or a composition thereof to induce apoptosis. The invention further provides treating lymphoma or Epstein-Barr virus (EBV) associated disease in a subject by administering Beta-Caryophyllene or a composition thereof.

### CROSS REFERENCE TO RELATED APPLICATIONS

This application claims priority to U.S. Provisional Patent Application 61/439,892, filed Feb. 6, 2011, which is incorporated by reference in its entirety.

### FIELD OF THE INVENTION

The invention relates to compositions and methods for inducing apoptosis in a cancer cell or a cell transformed by Epstein-Barr virus (EBV). Specifically, the invention relates to Beta-Caryophyllene or a composition thereof to induce apoptosis. The invention further relates to treating lymphoma or Epstein-Barr virus (EBV) associated disease in a subject by administering Beta-Caryophyllene or a composition thereof.

### BACKGROUND OF THE INVENTION

Beta-Caryophyllene (β-Caryophyllene, trans-(1R,9S)-8-Methylene-4,11,11-trimethylbicyclo[7.2.0]undec-4-ene or [1R-(1R,4E,9S)]-4,11,11-trimethyl-8-methylene-bicyclo[7.2.0]undec-4-ene) is a natural bicyclic sesquiterpene compound found in spice blends, citrus flavors, soaps, detergents, creams and lotions, and also in a variety of food products and beverages.

Beta-Caryophyllene has anti-inflammatory, local anesthetic activities, and anti-fungal activities. Beta-caryophyllene was shown to selectively bind the cannabinoid receptor type-2 and to exert significant cannabimimetic anti-inflammatory effects. Since the widespread plant natural product beta-caryophyllene is an FDA approved food additive and ingested daily with food. Beta-caryophyllene does not bind to the centrally expressed cannabinoid receptor type-1 (CB 1) and therefore does not exert psychomimetic effects.

Apoptosis (programmed cell death) is a frequent mode of cell death. Apoptosis is a natural phenomenon and occurs via a tightly regulated complex signaling cascade. A large number of drugs, both on the market and in development have apoptosis-modulating properties. In cancer and other diseases, elements of the apoptotic process become dysregulated, providing many molecular targets for drug discovery.

The primary site of Epstein-Barr virus (EBV) infection is the oropharyngeal cavity. Children and teenagers are commonly afflicted usually after oral contact, hence the name “kissing disease”. Based on serology, about 95% of the world adult population has been infected with EBV and, following primary infection, remains lifelong carriers of the virus. The disease is characterized by fever, sore throat, generalized lymphadenopathy, splenomegaly, intense asthenia, hyper-lymphocytosis (>50%) with atypical lymphocytes and elevated transaminase levels.

EBV is associated with Burkitt's B-cell lymphoma and nasopharyngeal carcinoma. According to the World Health Organization, Burkitt's lymphoma (BL) is a malignant form of tumor associated with EBV that is endemic to central parts of Africa and New Guinea with an annual incidence of 6-7 cases per 100 000 and a peak incidence at 6 or 7 years of age. The epidemiological involvement of EBV in Burkitt's lymphoma is based on the recognition of the EBV viral genome in tumor cells, associated with an elevated antibody titer against EBV viral capsid antigen (VCA). The highest prevalence of BL is found in the “lymphoma belt,” a region that extends from West to East Africa between the 10th degree north and 10th degree south of the equator and continues south down the Eastern coast of Africa. This area is characterized by high temperature and humidity, which is probably the reason why an association of malaria with BL was suspected at one time In African countries such as Uganda, in the lymphoma belt, the association of BL with EBV is very strong (97%), whereas it is weaker elsewhere (85% in Algeria; only 10-15% in France and the USA).

According to data from the World Health Organization, Nasopharyngeal cancer (NPC) incidence rates are less than 1 per 100,000 in most populations, except in populations in southern China, where an annual incidence of more than 20 cases per 100 000 is reported. Isolated northern populations such as Eskimos and Greenlanders also show high incidence. There is a moderate incidence in North Africa, Israel, Kuwait, the Sudan and parts of Kenya and Uganda. Men are twice as likely to develop NPC as women. The rate of incidence generally increases from ages 20 to around 50. In the USA, Chinese-Americans comprise the majority of NPC patients, together with workers exposed to fumes, smoke and chemicals, implying a role for chemical carcinogenesis.

### SUMMARY OF THE INVENTION

In one embodiment, the invention provides a method for inhibiting the proliferation of a cell transformed by Epstein-Barr virus (EBV), comprising the step of contacting said cell with an effective amount of a composition comprising Beta-Caryophyllene, thereby inhibiting the proliferation of said cell transformed by EBV.

In another embodiment, the invention provides a method of treating a subject infected by Epstein-Barr virus (EBV) or a subject having at least one cell transformed by EBV, comprising the step of administering to said subject an effective amount of a composition comprising Beta-Caryophyllene, thereby treating said subject infected by EBV or said subject having at least one cell transformed by EBV.

In another embodiment, the invention provides a method for inducing apoptosis in a cancer cell, comprising the step of contacting the cancer, cell with an effective amount of a composition comprising Beta-Caryophyllene.

In another embodiment, the invention provides a method of treating a subject afflicted with lymphoma, comprising the step of inducing apoptosis in a lymphoma cell in the subject, wherein inducing apoptosis in a lymphoma cell in the subject comprises administering to the subject an effective amount of a composition comprising Beta-Caryophyllene, thereby treating said subject afflicted with lymphoma.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

FIG. 1. is a bar graph showing the growth inhibitory effect of ethanol based Commiphora gileadensis stem extract on two tumor cell lines (concentrations in μl/mL, X axis). The cells were plated at concentration of 500,000 cells/mL and incubated with and without stem extract for 17 h. Control cells were treated with ethanol (0.05%) alone.

FIG. 2. is a bar graph showing the growth inhibitory effect of C. gileadensis essential oil on two tumor cell lines. The cells were plated at concentration of 500,000 cells/mL and incubated with citral (3.1e-4 μM), STS (2 μM) and three concentrations of essential oil from C. gileadensis for 2 hours (h). Control cells were treated with ethanol (0.05%) alone.

FIG. 3. is a bar graph showing the growth inhibitory effect of β-Caryophyllene on two tumor cell lines. The cells were plated at concentration of 500,000 cells/mL and incubated with citral (3.1e-4 μM), STS (2 μM) and three β-caryophyllene concentrations (2.4e⁻⁴, 4.8e⁻⁴, and 9.6e⁻⁴ μM) for 2 h. Control cells were treated with ethanol (0.05%) alone.

FIG. 4. is a bar graph showing the apoptogenic effects of β-Caryophyllene on two tumor cell lines. Enzymatic activity of different cell lines incubated with β-Caryophyllene (2.4e⁻⁴ μM) for 2 h. X axis represents the time of the enzymatic reaction in vitro in minutes.

FIG. 5. is a bar graph showing the apoptogenic effects of β-Caryophyllene on two tumor cell lines. Enzymatic activity of different cell lines incubated with Caryophyllene (2.4e⁻⁴ μM) for 2 hrs. Measurements were taken following 1020 min of enzymatic reaction in vitro. Pre-incubation with caspase-3 inhibitor (DEVD-CHO) eliminated any development of caspase-3 activity.

FIG. 6. is a gel micrograph showing the DNA ladder effect (induction of apoptosis) of different compositions. BS-24-1 cells (1,000,000/mL) were incubated with C. gileadensis stem and leaf extracts (6 μl/mL) for 17 h. DNA was separated and analyzed on agarose gel. Lanes 1-7 and 11, H₂O based stem extract. Lanes 8-10, H₂O based leaf extract. Lanes 12-18 and 22, ethanol based stem extract. Lanes 19-21, ethanol based leaf extract. Lanes 23 and 24 Control cells treated with ethanol (0.05%) and water.

FIG. 7. is a gel micrograph showing the DNA ladder effect (induction of apoptosis) of different compositions. MoFir and normal FB cells were incubated with C. gileadensis extract (2.5 μl/mL) for 24 h. DNA was separated and analyzed on agarose gel. Lane 1 and 2, ethanol based stem extract Lane 3-5, ethanol based leaf extract. Lane 6 control cells treated with ethanol (0.05%).

FIG. 8. is a gel micrograph showing the DNA ladder effect (induction of apoptosis) of different compositions. BS-24-1 cells were incubated with C. gileadensis essential oil for 24 h. DNA was separated and analyzed on agarose gel. Lane 1, untreated cells with ethanol (0.05%) alone. Lane 2, cells incubation with 0.625 μl/mL essential oil. Lane 3, cells incubation with 1.25 μl/mL essential oil.

FIG. 9. is a gel micrograph showing the DNA ladder effect (induction of apoptosis) of β-Caryophyllene. Fragmentation of DNA in the presence of β-Caryophyllene. BS-24-1 cells were incubated in the presence of β-Caryophyllene in three different concentrations and two incubation time Lane 1, untreated cells with ethanol (0.05%) alone. Lane 2, cells incubated for 24 h with 2.4 μl M Lane 3, cells incubated for 2 h with 9.6 μM β-caryophyllene.

FIG. 10 shows the growth inhibitory effect of β-Caryophyllene on B95 cells (i.e., producer of EBV).

FIG. 11 shows that β-Caryophyllene inhibits the activity of Topoisomerase I.

FIG. 12 shows that β-Caryophyllene inhibits the activity of Topoisomerase I.

FIG. 13 demonstrates that β-Caryophyllene inhibits the activity of NF-kB based on a luciferase assay.

### DETAILED DESCRIPTION OF THE INVENTION

The invention provides compositions and methods for inducing apoptosis in a cancer cell or a cell transformed by Epstein-Barr virus (EBV). Specifically, the invention provides Beta-Caryophyllene or a composition thereof to induce apoptosis. The invention further provides treating lymphoma or an Epstein-Barr Virus (EBV)-associated disease in a subject by administering Beta-Caryophyllene or a composition thereof.

In one embodiment, provided herein is a method for inducing apoptosis in a cancer cell, comprising the step of contacting the cell with an effective amount of a composition comprising Beta-Caryophyllene. In another embodiment, provided herein is a method for inducing apoptosis and not necrosis in a cancer cell, comprising the step of contacting the cell with an effective amount of a composition comprising Beta-Caryophyllene. In another embodiment, provided herein is a method for selectively inducing apoptosis in a cancer cell present in an environment comprising both cancer and normal cells. In another embodiment, the phrase “normal cell” includes a non-cancer cell, a cell free of viral infections, a cell free of any intracellular pathogens, a differentiated cell, or any combination thereof. In another embodiment, the present invention provides a method wherein inducing apoptosis in a cancer cell comprises differentially inducing apoptosis in a cancer cell over a non-cancer cell. In another embodiment, the present invention provides that inducing apoptosis in a cancer cell comprises activating caspase 3.

In another embodiment, provided herein is a method for inducing apoptosis in a cell infected and/or transformed by Epstein-Barr virus (EBV), comprising the step of contacting the cell with an effective amount of a composition comprising Beta-Caryophyllene. In another embodiment, provided herein is a method for inducing apoptosis and not necrosis in a cell infected and/or transformed by Epstein-Barr virus (EBV), comprising the step of contacting the cell with an effective amount of a composition comprising Beta-Caryophyllene. In another embodiment, provided herein is a method for selectively inducing apoptosis in a cell infected and/or transformed by Epstein-Barr virus (EBV) present in an environment comprising both cells infected and/or transformed by Epstein-Barr virus (EBV) and normal cells. In another embodiment, the phrase “normal cell” includes a non-cancer cell, a cell free of viral infections, a cell free of any intracellular pathogens, a differentiated cell, or any combination thereof.

In another embodiment, the phrase “an effective amount of a composition comprising Beta-Caryophyllene” means the amount of a composition comprising Beta-Caryophyllene that selectively induces apoptosis in cancerous cells or cells infected and/or transformed by Epstein-Barr virus (EBV). In another embodiment, the phrase “an effective amount of a composition comprising Beta-Caryophyllene” means the amount of a composition comprising Beta-Caryophyllene that selectively induces apoptosis in cancerous cells or cells infected and/or transformed by EBV present in an environment comprising normal cells. In another embodiment, an environment comprising normal cells is a tissue. In another embodiment, an environment comprising normal cells is an organ. In another embodiment, an environment comprising normal cells is a human or an animal body. In another embodiment, an environment comprising normal cells also comprises cells infected and/or transformed by EBV and/or cancer cell.

In another embodiment, the term “selectively” includes increased potency. In another embodiment, the term “selectively” is synonymous with differentially. In another embodiment, the term “selectively” means specifically.

In another embodiment, the term “selectively” indicates a 10-fold potency for one cell type over another. In another embodiment, a composition of the invention that selectively induces apoptosis in cancerous cells or cells infected and/or transformed by EBV is a composition that is at least 5 times more effective in inducing apoptosis in cancerous cells or cells infected and/or transformed by EBV compared to normal cells. In another embodiment, a composition of the invention that selectively induces apoptosis in cancerous cells or cells infected and/or transformed by EBV is a composition that is at least 10 times more effective in inducing apoptosis in cancerous cells or cells infected and/or transformed by EBV compared to normal cells. In another embodiment, a composition of the invention that selectively induces apoptosis in cancerous cells or cells infected and/or transformed by EBV is a composition that is at least 15 times more effective in inducing apoptosis in cancerous cells or cells infected and/or transformed by EBV compared to normal cells. In another embodiment, a composition of the invention that selectively induces apoptosis in cancerous cells or cells infected and/or transformed by EBV is a composition that is at least 20 times more effective in inducing apoptosis in cancerous cells or cells infected and/or transformed by EBV compared to normal cells. In another embodiment, a composition of the invention that selectively induces apoptosis in cancerous cells or cells infected and/or transformed by EBV is a composition that is at least 30 times more effective in inducing apoptosis in cancerous cells or cells infected and/or transformed by EBV compared to normal cells. In another embodiment, a composition of the invention that selectively induces apoptosis in cancerous cells or cells infected and/or transformed by EBV is a composition that is at least 50 times more effective in inducing apoptosis in cancerous cells or cells infected and/or transformed by EBV compared to normal cells.

In another embodiment, cell or cells of the invention including cancerous cells and cells infected and/or transformed by EBV are human or monkey cells. In another embodiment, cell or cells of the invention including cancerous cells and cells infected and/or transformed by EBV are rodent cells. In another embodiment, cell or cells of the invention including cancerous cells and cells infected and/or transformed by EBV are human or monkey derived cells. In another embodiment, cell or cells of the invention including cancerous cells and cells infected and/or transformed by EBV are rodent derived cells.

In another embodiment, Beta-Caryophyllene is extracted from a plant. In another embodiment, Beta-Caryophyllene is present with an oil extract derived from a plant. In another embodiment, a composition as described herein which comprises Beta-Caryophyllene is an oil extract derived from a. plant. In another embodiment, a composition as described herein which comprises Beta-Caryophyllene is a plant extract. In another embodiment, a composition as described herein which comprises Beta-Caryophyllene comprises a plant extract. In another embodiment, the plant is Commiphora gileadensis.

In one embodiment, beta-caryophyllene is obtained from Commiphora gileadensis. In another embodiment, beta-caryophyllene is obtained from Cannabis, hemp, marijuana (Cannabis sativa); Black Caraway (Carum nigrum); Cloves (Syzygium aromaticum); Hops (Humulus lupulus); Basil (Ocimum spp.); Oregano (Origanum vulgare); Black pepper (Piper nigrum); West African Pepper (Piper guineense); Rosemary (Rosmarinus officinalis); True cinnamon (Cinnamomum zeylanicum); or Malabathrum (Cinnamomum tamala).

In one embodiment, the method of the present invention are used to treat or prevent a cancer. In one embodiment, the cancer is lymphoma. In another embodiment, the cancer is nasopharyngeal carcinoma. In another embodiment, the cancer is Hodgkin's lymphoma (HL). In another embodiment, the cancer is T-cell lymphoma. In another embodiment, the cancer is an epithelial tumor such as gastric cancer. In another embodiment, the cancer is any cancer characterized by the presence of multiple extra-chromosomal copies of the EBV viral genome in tumor cells and the expression of part of the EBV genome. In another embodiment, the cancer is Burkitt's lymphoma. In another embodiment, the cancer is a central nervous system lymphoma associated with HIV.

Thus, in one embodiment, the present invention provides a method of treating or preventing Burkitt's lymphoma. In another embodiment, the present invention provides a method of treating or preventing nasopharyngeal cancer.

In one embodiment, the present invention provides a method of treating or preventing a cancer. In another embodiment, the present invention provides a method of treating or preventing a tumor. In one embodiment, the present invention provides a method of treating or preventing a cancer by inducing apoptosis in a cancer cell. In one embodiment, the present invention provides a method of treating or preventing infection of a subject with EBV by inducing apoptosis in a cell infected with EBV.

In one embodiment, Beta-Caryophyllene inhibits infection of a cell with EBV. In another embodiment, Beta-Caryophyllene inhibits EBV proliferation. In one embodiment, administration of Beta-Caryophyllene to a subject prevents symptoms of EBV infection, in one embodiment, by inhibiting the spread of EBV, or in another embodiment, inhibiting infection of cells with EBV, or in another embodiment, inhibiting EBV proliferation.

In another embodiment, the compositions and methods of the present invention may be used to treat an autoimmune disease associated with EBV, which in one embodiment, is dermatomyositis, systemic lupus erythematosus, rheumatoid arthritis, Sjögren's syndrome, and multiple sclerosis, or a combination thereof. In one embodiment, EBV is referred to as human herpesvirus 4 (HHV-4).

In another embodiment, a cell infected and/or transformed by EBV is a tumor cell. In another embodiment, a cell infected and/or transformed by EBV is a B-cell lymphoma cell. In another embodiment, a cell infected and/or transformed by EBV is a Burkitt's lymphoma cell. In another embodiment, a cell transformed by EBV is a nasopharyngeal carcinoma cell. In another embodiment, a cell infected and/or transformed by EBV is an epithelial cell. In another embodiment, a cell infected and/or transformed by EBV is an oral epithelial cell. In another embodiment, a cell infected and/or transformed by EBV is an oro-pharyngeal epithelial cell. In another embodiment, a cell infected and/or transformed by EBV is a NK cell. In another embodiment, a cell infected and/or transformed by EBV is a T-cell. In another embodiment, a cell infected and/or transformed by EBV is a Hodgkin's lymphoma (HL) cell. In another embodiment, a cell infected and/or transformed by EBV is a T-cell lymphoma cell.

In another embodiment, provided herein a method for, treating a subject afflicted with lymphoma, comprising the step of inducing apoptosis in a lymphoma cell in the subject, wherein inducing apoptosis in a lymphoma cell in the subject comprises administering to the subject an effective amount of a composition comprising Beta-Caryophyllene. In another embodiment, inducing apoptosis in a lymphoma cell in the subject does not include induction of necrosis.

In another embodiment, lymphoma is an indolent lymphoma. In another embodiment, lymphoma is Burkitt's lymphoma. In another embodiment, lymphoma is a mature B cell neoplasm. In another embodiment, lymphoma is diffuse large B-cell lymphoma. In another embodiment, lymphoma is B-cell prolymphocytic leukemia. In another embodiment, lymphoma is Lymphoplasmacytic lymphoma. In another embodiment, lymphoma is splenic marginal zone lymphoma. In another embodiment, lymphoma is a plasma cell neoplasm. In another embodiment, lymphoma is a plasma cell myeloma. In another embodiment, lymphoma is plasmacytoma. In another embodiment, lymphoma is MALT lymphoma. In another embodiment, lymphoma is follicular lymphoma. In another embodiment, lymphoma is mantle cell lymphoma. In another embodiment, lymphoma is mediastinal (thymic) large B cell lymphoma. In another embodiment, lymphoma is intravascular large B cell lymphoma. In another embodiment, lymphoma is primary effusion lymphoma. In another embodiment, lymphoma is T cell large granular lymphocytic leukemia. In another embodiment, lymphoma is aggressive NK cell leukemia. In another embodiment, lymphoma is adult T cell leukemia. In another embodiment, lymphoma is adult T cell lymphoma. In another embodiment, lymphoma is Extranodal NK. In another embodiment, lymphoma is T cell lymphoma.

In another embodiment, a method for treating a subject afflicted with lymphoma is a method of inhibiting lymphoma. In another embodiment, a method for treating a subject afflicted with lymphoma is a method of eliminating lymphoma cells in a subject. In another embodiment, a method for treating a subject afflicted with lymphoma is a method of eliminating lymphoma cells in a subject but not normal cells. In another embodiment, a method for treating a subject afflicted with lymphoma is a method for specifically and/or differentially eliminating lymphoma cells over normal cells. In another embodiment, a method for treating a subject afflicted with lymphoma is a method of curing lymphoma.

In another embodiment, a method for treating a subject afflicted with lymphoma is a method of inhibiting the growth of a solid tumor of lymphoid cells. In another embodiment, a method for treating a subject afflicted with lymphoma as described herein further comprises additionally administering to the subject other means of chemotherapy known to be effective in treating lymphoma. In another embodiment, a method for treating a subject afflicted with lymphoma as described herein further comprises radiating the subject. In another embodiment, a method for treating a subject afflicted with lymphoma comprises administering the composition of the invention before, during, and/or after bone marrow transplantation for the elimination of remaining lymphoma cells.

In another embodiment, a method for treating a subject afflicted with lymphoma comprises administering the composition of the invention directly to the blood (such as IV administration). In another embodiment, a method for treating a subject afflicted with lymphoma comprises administering the composition of the invention directly to a lymph node comprising lymphoma cells. In another embodiment, a method for treating a subject afflicted with lymphoma is a method of treating extranodal lymphoma comprising administering the composition of the invention directly to an extranodal site such as but not limited to the skin, brain, bowels and bone.

In another embodiment, the present provides a method of treating a subject infected by Epstein-Barr virus (EBV) or comprising at least one cell transformed by EBV, comprising the step of administering to the subject an effective amount of a composition comprising Beta-Caryophyllene. In another embodiment, a subject infected by Epstein-Barr virus (EBV) is a subject comprising a cell infected by EBV or a subject comprising at least one cell transformed by EBV. In another embodiment, a subject infected by EBV is a subject comprising a cell that was previously infected by EBV and having at least one cell transformed by EBV. In another embodiment, a subject infected by EBV is a subject comprising a cell that was previously infected by EBV and comprises extrachromosomal copies of the EBV viral genome. In another embodiment, a subject having extrachromosomal copies of the EBV viral genome in at leasy one cell is a subject comprising at least one cell transformed by EBV.

In another embodiment, the invention provides a method for inhibiting the proliferation of a cell producing Epstein-Barr virus (EBV), the method comprising: providing an effective amount of Beta-Caryophyllene to said cell, thereby inhibiting the proliferation of a cell producing EBV.

In another embodiment, the invention provides a method for inhibiting the activity of Topoisomerase I in a cell, the method comprising: providing an effective amount of Beta-Caryophyllene to said cell, thereby inhibiting the activity of Topoisomerase I in said cell.

In another embodiment, the invention provides a method for inhibiting the activity of Nuclear factor kappa B (NF-kB) in a cell, the method comprising: providing an effective amount of Beta-Caryophyllene to said cell, thereby inhibiting the activity of NF-kB in said cell.

In another embodiment, the invention provides a method for inhibiting the replication of Epstein-Barr virus (EBV) in a cell, the method comprising: providing an effective amount of Beta-Caryophyllene to said cell, thereby inhibiting the replication of Epstein-Barr virus (EBV) in said cell.

In another embodiment, the invention provides a method for treating a disease associated with Epstein-Barr virus (EBV), the method comprising: providing an effective amount of Beta-Caryophyllene to a cell or subject in need thereof, wherein Beta-Caryophyllene inhibits the replication of EBV by inhibiting the activity of Topoisomerase I, Nuclear factor kappa 13 (NF-kB), or a combination thereof, thereby said method treats said disease associated with EBV.

In another embodiment, a composition comprising Beta-Caryophyllene is unexpectedly effective in a treatment according to the invention because it induces the elimination of cells infected with EBV and/or cells that were previously infected by EBV and comprise extrachromosomal copies of the EBV viral genome. In another embodiment, a composition comprising Beta-Caryophyllene is unexpectedly effective in a treatment according to the invention because it induces apoptosis in cells infected with EBV and/or cells that were previously infected by EBV and comprise extrachromosomal copies of the EBV viral genome. In another embodiment, inducing apoptosis in cells infected with EBV and/or cells that were previously infected by EBV is differentially and/or selectively inducing apoptosis in cells infected with EBV and/or cells that were previously infected by EBV over a non-cancer cell or a non-EBV infected and/or transformed cell. In another embodiment, Beta-Caryophyllene according to the invention is unexpectedly toxic to cells infected with EBV and/or cells that were previously infected by EBV and comprise extrachromosomal copies of the EBV viral genome.

In another embodiment, a composition comprising Beta-Caryophyllene is unexpectedly effective in a treatment according to the invention because it specifically and/or differentially induces the elimination and/or apoptosis of cells infected with EBV and/or cells that were previously infected by EBV and comprise extrachromosomal copies of the EBV viral genome but not of normal cells. In another embodiment, a composition comprising Beta-Caryophyllene is unexpectedly effective in a treatment according to the invention because it specifically and/or differentially induces the elimination and/or apoptosis of cells infected with EBV and/or cells that were previously infected by EBV but not of normal cells that are not infected and/or comprise extrachromosomal copies of the EBV viral genome. In another embodiment, a composition comprising Beta-Caryophyllene is unexpectedly effective in a treatment according to the invention because it specifically and/or differentially induces the elimination and/or apoptosis of cancer cells, cells infected with EBV and/or cells that were previously infected by EBV but not of normal cells.

In another embodiment, a subject infected with EBV is afflicted with infectious mononucleosis. In another embodiment, treating a subject infected by EBV is inhibiting a disease that is caused by an EBV such as lymphoma. In another embodiment, treating a subject infected by EBV is inhibiting and/or slowing the progression of a disease that is caused by an EBV such as lymphoma. In another embodiment, treating a subject infected by EBV is reducing the risk for the progression of a disease that is caused by an EBV such as lymphoma. In another embodiment, treating a subject infected by EBV is curing a disease that is caused by an EBV such as lymphoma. In another embodiment, treating a subject infected by EBV is ameliorating symptoms associated with a disease that is caused by an EBV such as lymphoma.

In another embodiment, treating a subject infected by EBV is eliminating cells that are infected or were previously infected by an EBV. In another embodiment, treating a subject infected by EBV is reducing the number of cells that are infected or were previously infected by an EBV. In another embodiment, treating a subject infected by EBV is treating a subject afflicted with EBV associated cancer such as mentioned herein. In another embodiment, treating a subject infected by EBV is preventing persistence of EBV in the bone marrow. In another embodiment, treating a subject infected by EBV is preventing and/or inhibiting latent infection of B-lymphocytes. In another embodiment, treating a subject infected by EBV is reducing a risk of a malignancy. In another embodiment, treating a subject infected by EBV is reducing a risk of a malignancy associated or induced by EBV.

In another embodiment, treating a subject infected by EBV is inhibiting or reducing the risk of a persistent infection by EBV. In another embodiment, treating a subject infected by EBV is inhibiting or reducing the risk of a latent infection by EBV. In another embodiment, treating a subject infected by EBV is eliminating or inducing apoptosis in a β-lymphocyte infected by EBV or a B-lymphocyte that was previously infected by EBV as described herein. In another embodiment, inducing apoptosis in a B-lymphocyte infected by EBV or a B-lymphocyte that was previously infected by EBV is inducing apoptosis in a proliferating B-lymphocyte. In another embodiment, there is a long felt need for the method of the invention which can treat a subject infected with EBV prior to the stage wherein immunity develops for EBV.

In some embodiments, providing an effective amount of Beta-Caryophyllene inhibits the proliferation of a cell producing EBV. In other embodiments, providing an effective amount of Beta-Caryophyllene inhibits the replication of EBV by inhibiting the activity of Topoisomerase I, Nuclear factor kappa B (NF-kB), or a combination thereof. In one embodiment, the invention provides a method for treating a disease associated with EBV, the method comprising: providing an effective amount of Beta-Caryophyllene to a cell or subject in need thereof, wherein Beta-Caryophyllene inhibits the replication of EBV by inhibiting the activity of Topoisomerase I, Nuclear factor kappa B (NF-kB), or a combination thereof, thereby said method treats said disease associated with EBV.

In another embodiment, beta-Caryophyllene is administered in a dose of 1-90 micrograms in 0.1-5 ml solution (composition as described herein). In another embodiment, beta-Caryophyllene is administered in a dose of 1-50 micrograms in 0.1-5 ml solution. In another embodiment, beta-Caryophyllene is administered in a dose of 1-25 micrograms in 0.1-5 ml solution. In another embodiment, beta-Caryophyllene is administered in a dose of 50-90 micrograms in 0.1-5 ml solution. In another embodiment, beta-Caryophyllene is administered in a dose of 10-50 micrograms in 0.1-5 ml solution.

In another embodiment, beta-Caryophyllene is administered in a dose of 0.01-10 grams. In another embodiment, beta-Caryophyllene is administered in a dose of 0.01-0.1 grams. In another embodiment, beta-Caryophyllene is administered in a dose of 0.1-1 grams. In another embodiment, beta-Caryophyllene is administered in a dose of 1-5 grams. In another embodiment, beta-Caryophyllene is administered in a dose of 5-10 grams. In another embodiment, beta-Caryophyllene is administered in a dose of 10-50 grams.

In another embodiment, beta-Caryophyllene is administered in a dose of 0.01-100 milligrams per kilogram body weight. In another embodiment, beta-Caryophyllene is administered in a dose of 0.01-0.1 milligrams per kilogram body weight. In another embodiment, beta-Caryophyllene is administered in a dose of 0.1-1 milligrams per kilogram body weight. In another embodiment, beta-Caryophyllene is administered in a dose of 1-5 milligrams per kilogram body weight. In another embodiment, beta-Caryophyllene is administered in a dose of 1-10 milligrams per kilogram body weight. In another embodiment, beta-Caryophyllene is administered in a dose of 1-100 milligrams per kilogram body weight. In another embodiment, beta-Caryophyllene is administered in a dose of 20-50 milligrams per kilogram body weight. In another embodiment, beta-Caryophyllene is administered in a dose of 40-100 milligrams per kilogram body weight.

In another embodiment, a composition of the invention comprises beta-caryophyllene and a pharmaceutically acceptable carrier. In another embodiment, a composition of the invention comprising beta-caryophyllene is administered by an intramuscular (IM) injection, a subcutaneous (SC) injection, or an intravenous (IV) injection.

In another embodiment, a composition of the invention is a “pharmaceutical composition”. In another embodiment, a “pharmaceutical composition” refers to a preparation of at least beta-Caryophyllene with other chemical components such as physiologically suitable carriers and excipients. The purpose of a pharmaceutical composition is to facilitate administration of a compound to an organism.

In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In another embodiment, the phrases “physiologically acceptable carrier” and “pharmaceutically acceptable carrier” which be interchangeably used refer to a carrier or a diluent that does not cause significant irritation to an organism and does not abrogate the biological activity and properties of the administered compound. An adjuvant is included under these phrases. In one embodiment, one of the ingredients included in the pharmaceutically acceptable carrier can be for example polyethylene glycol (PEG), a biocompatible polymer with a wide range of solubility in both organic and aqueous media (Mutter et al. (1979).

In another embodiment, “excipient” refers to an inert substance added to a pharmaceutical composition to further facilitate administration of an active ingredient. In one embodiment, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences,” Mack Publishing Co., Easton, Pa., latest edition, which is incorporated herein by reference.

In another embodiment, suitable routes of administration, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

In another embodiment, the preparation is administered in a local rather than systemic manner, for example, via injection of the preparation directly into a specific region of a patient's body.

Various embodiments of dosage ranges are contemplated by this invention. The dosage of beta-caryophyllene, in one embodiment, is in the range of 0.005-100 mg/day. In another embodiment, the dosage is in the range of 0.005-5 mg/day. In another embodiment, the dosage is in the range of 0.01-50 mg/day. In another embodiment, the dosage is in the range of 0.1-20 mg/day. In another embodiment, the dosage is in the range of 0.1-10 mg/day. In another embodiment, the dosage is in the range of 0.01-5 mg/day. In another embodiment, the dosage is in the range of 0.001-0.01 mg/day. In another embodiment, the dosage is in the range of 0.001-0.1 mg/day. In another embodiment, the dosage is in the range of 0.1-5 mg/day. In another embodiment, the dosage is in the range of 0.5-50 mg/day. In another embodiment, the dosage is in the range of 0.2-15 mg/day. In another embodiment, the dosage is, in the range of 0.8-65 mg/day. In another embodiment, the dosage is in the range of 1-50 mg/day. In another embodiment, the dosage is in the range of 5-10 mg/day. In another embodiment, the dosage is in the range of 8-15 mg/day. In another embodiment, the dosage is in a range of 10-20 mg/day. In another embodiment, the dosage is in the range of 20-40 mg/day. In another embodiment, the dosage is in a range of 60-120 mg/day. In another embodiment, the dosage is in the range of 12-40 mg/day. In another embodiment, the dosage is in the range of 40-60 mg/day. In another embodiment, the dosage is in a range of 50-100 mg/day. In another embodiment, the dosage is in a range of 1-60 mg/day. In another embodiment, the dosage is in the range of 15-25 mg/day. In another embodiment, the dosage is in the range of 5-10 mg/day. In another embodiment, the dosage is in the range of 55-65 mg/day.

In another embodiment, beta-caryophyllene is formulated in an oral dosage form. In another embodiment, beta-caryophyllene is formulated in a peroral dosage form. In another embodiment, beta-caryophyllene is formulated in an intranasal dosage form. In another embodiment, beta-caryophyllene is formulated in an injectable dosage form.

Oral administration, in one embodiment, comprises a unit dosage form comprising tablets, capsules, lozenges, chewable tablets, suspensions, emulsions and the like. Such unit dosage forms comprise a safe and effective amount of beta-caryophyllene. The pharmaceutically-acceptable carriers suitable for the preparation of unit dosage forms for peroral administration are well-known in the art. In some embodiments, tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. In one embodiment, glidants such as silicon dioxide can be used to improve flow characteristics of the powder-mixture. In one embodiment, coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. In some embodiments, the selection of carrier components depends on secondary considerations like taste, cost, and shelf stability, which are not critical for the purposes of this invention, and can be readily made by a person skilled in the art.

In one embodiment, the oral dosage form comprises predefined release profile. In one embodiment, the oral dosage form of the present invention comprises an extended release tablets, capsules, lozenges or chewable tablets. In one embodiment, the oral dosage form of the present invention comprises a slow release tablets, capsules, lozenges or chewable tablets. In one embodiment, the oral, dosage form of the present invention comprises an immediate release tablets, capsules, lozenges or chewable tablets. In one embodiment, the oral dosage form is formulated according to the desired release profile of the pharmaceutical active ingredient as known to one skilled in the art.

Peroral compositions, in some embodiments, comprise liquid solutions, emulsions, suspensions, and the like. In some embodiments, pharmaceutically-acceptable carriers suitable for preparation of such compositions are well known in the art. In some embodiments, liquid oral compositions comprise from about 0.001% to about 0.933% of the desired compound or compounds, or in another embodiment, from about 0.01% to about 10%.

In some embodiments, compositions for use in the methods of this invention comprise solutions or emulsions, which in some embodiments are aqueous solutions or emulsions comprising a safe and effective amount of the compounds of the present invention and optionally, other compounds, intended for topical intranasal administration. In some embodiments, h compositions comprise from about 0.001% to about 10.0% w/v of a subject compound, more preferably from about 00.1% to about 2.0, which is used for systemic delivery of the compounds by the intranasal route.

In another embodiment, the pharmaceutical compositions are administered by intravenous, intra-arterial, or intramuscular injection of a liquid preparation. In some embodiments, liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration. In another embodiment, the pharmaceutical compositions are administered intramuscularly, and are thus formulated in a form suitable for intramuscular administration.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, the compounds of the present invention are combined with an additional appropriate therapeutic agent or agents, prepared and applied as solutions, suspensions, or emulsions in a physiologically acceptable diluent with or without a pharmaceutical carrier.

In one embodiment, pharmaceutical compositions of the present invention are manufactured by processes well known in the art, e.g., by means of conventional mixing, dissolving, granulating, dragee-making, levigating, emulsifying, encapsulating, entrapping or lyophilizing processes.

In one embodiment, pharmaceutical compositions for use in accordance with the present invention is formulated in conventional manner using one or more physiologically acceptable carriers comprising excipients and auxiliaries, which facilitate processing of the active ingredients into preparations which, can be used pharmaceutically. In one embodiment, formulation is dependent upon the route of administration chosen.

In one embodiment, injectables, of the invention are formulated in aqueous solutions. In one embodiment, injectables, of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, compositions are suspensions, solutions or emulsions in oily or aqueous vehicles, and contain formulatory agents such as suspending, stabilizing and/or dispersing agents.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The compositions also comprise, in some embodiments, local anesthetics or other actives. The compositions can be used as sprays, mists, drops, and the like.

In some embodiments, pharmaceutical compositions for parenteral administration include aqueous solutions of the active preparation in water-soluble form. Additionally, suspensions of the active ingredients, in some embodiments, are prepared as appropriate oily or water based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances, which increase the viscosity of the suspension, such as sodium carboxymethyl cellulose, sorbitol or dextran. In another embodiment, the suspension also contain suitable stabilizers or agents which increase the solubility of the active ingredients to allow for the preparation of highly concentrated solutions.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989), Lopez-Berestein, ibid., pp. 317-327; see generally ibid).

In another embodiment, the pharmaceutical composition delivered in a controlled release system is formulated for intravenous infusion, implantable osmotic pump, transdermal patch, liposomes, or other modes of administration. In one embodiment, a pump is used (see Langer, supra; Sefton, CRC Crit. Ref Biomed Eng 14:201 (1987); Buchwald et al., Surgery 88:507 (1980); Saudek et al., N Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

In some embodiments, the active ingredient is in powder form for constitution with a suitable vehicle, e.g., sterile, pyrogen-free water based solution, before use Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

In some embodiments, pharmaceutical compositions suitable for use in context of the present invention include compositions wherein the active ingredients are contained in an amount effective to achieve the intended purpose. In some embodiments, a therapeutically effective amount means an amount of active ingredients effective to prevent, alleviate or ameliorate symptoms of disease or prolong the survival of the subject being treated.

In one embodiment, determination of a therapeutically effective amount is well within the capability of those skilled in the art.

Some examples of substances which can serve as pharmaceutically-acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the compound is basically determined by the way the compound is to be administered. If the subject compound is to be injected, in one embodiment, the pharmaceutically-acceptable carrier is sterile, physiological saline, with a blood-compatible suspending agent, the pH of which has been adjusted to about 7.4.

In addition, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, cellulose (e.g. Avicel™, RC-591), tragacanth and sodium alginate; typical wetting agents include lecithin and polyethylene oxide sorbitan (e.g. polysorbate 80). Typical preservatives include methyl paraben and sodium benzoate. In another embodiment, peroral liquid compositions also contain one or more components such as sweeteners, flavoring agents and colorants disclosed above.

The compositions also include incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In some embodiments, compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. In another embodiment, the modified compounds exhibit substantially longer half-lives in blood following intravenous injection than do the corresponding unmodified compounds. In one embodiment, modifications also increase the compound's solubility in aqueous solution, eliminate aggregation, enhance the physical and chemical stability of the compound, and greatly reduce the immunogenicity and reactivity of the compound. In another embodiment, the desired in vivo biological activity is achieved by the administration of such polymer-compound abducts less frequently or in lower doses than with the unmodified compound.

In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

In one embodiment, depending on the severity and responsiveness of the condition to be treated, dosing can be of a single or a plurality of administrations, with course of treatment lasting from several days to several weeks or until cure is effected or diminution of the disease state is achieved.

In one embodiment, the amount of a composition to be administered will, of course, be dependent on the subject being treated, the severity of the affliction, the manner of administration, the judgment of the prescribing physician, etc.

In one embodiment, compositions including the preparation of the present invention formulated in a compatible pharmaceutical carrier are also be prepared, placed in an appropriate container, and labeled for treatment of an indicated condition.

In another embodiment, beta-caryophyllene is administered via systemic administration. In another embodiment, beta-caryophyllene is administered by intravenous, intramuscular or subcutaneous injection. In another embodiment, beta-caryophyllene is lyophilized (i.e., freeze-dried) preparation in combination with complex organic excipients and stabilizers such as nonionic surface active agents (i.e., surfactants), various sugars, organic polyols and/or human serum albumin.

In another embodiment, the pharmaceutical composition comprises beta-caryophyllene and complex carriers such as human serum albumin, polyols, sugars, and anionic surface active stabilizing agents. See, for example, WO 89/10756 (Hara et al.—containing polyol and p-hydroxybenzoate). In another embodiment, the pharmaceutical composition comprises beta-caryophyllene and lactobionic acid and an acetate/glycine buffer. In another embodiment, the pharmaceutical composition comprises beta-caryophyllene and amino acids, such as arginine or glutamate that increase the solubility of interferon compositions in water.

In another embodiment, the pharmaceutical composition comprising beta-caryophyllene is stabilized when placed in buffered solutions having a pH between about 4 and 7.2. In another embodiment, the pharmaceutical composition comprising beta-caryophyllene is stabilized with an amino acid as a stabilizing agent and in some cases a salt (if the amino acid does not contain a charged side chain).

In another embodiment, the pharmaceutical composition comprising beta-caryophyllene is a liquid composition comprising a stabilizing agent at between about 0.3% and 5% by weight which is an amino acid.

In another embodiment, the pharmaceutical composition comprising beta-caryophyllene provides dosing accuracy and product safety. In another embodiment, the pharmaceutical composition comprising beta-caryophyllene provides a biologically active, stable liquid formulation for use in injectable applications.

In another embodiment, the pharmaceutical composition comprising beta-caryophyllene provides a liquid formulation permitting storage for a long period of time in a liquid state facilitating storage and shipping prior to administration.

In another embodiment, the pharmaceutical composition comprising beta-caryophyllene comprises solid lipids as matrix material. In another embodiment, the injectable pharmaceutical composition comprising beta-caryophyllene comprises solid lipids as matrix material. In another embodiment, the production of lipid microparticles by spray congealing was described by Speiser (Speiser and al., Pharma Res. 8 (1991) 47-54) followed by lipid nanopellets for peroral administration (Speiser EP 0167825 (1990)). In another embodiment, lipids, which are used, are well tolerated by the body (e.g. glycerides composed of fatty acids which are present in the emulsions for parenteral nutrition).

In another embodiment, the pharmaceutical composition comprising beta-caryophyllene is in the form of liposomes (J. E. Diederichs and al., Pharm. /nd. 56 (1994) 267-275).

In another embodiment, the composition is a plant extract. In another embodiment, the composition is an extract of oil from a plant. In another embodiment, the composition is a Commiphpora gileadensis extract. In another embodiment, the composition is a food additive.

In another embodiment, the invention provides, a composition comprising Beta-Caryophyllene in an amount effective to induce apoptosis in a cancer cell.

In another embodiment, the invention provides a composition comprising Beta-Caryophyllene in an amount effective to induce apoptosis in a cell transformed by Epstein-Barr virus (EBV).

In another embodiment the invention provides a composition comprising Beta-Caryophyllene in an amount effective to inhibit the proliferation of a cell producing Epstein-Barr virus (EBV).

In another embodiment, the invention provides a composition comprising Beta-Caryophyllene in an amount effective to inhibit the activity of Nuclear factor kappa B (NF-kB) or Topoisomerase I in order to inhibit the replication of Epstein-Barr virus (EBV).

In another embodiment, the pharmaceutical composition comprising beta-caryophyllene comprises polymeric microparticles. In another embodiment, the injectable pharmaceutical composition comprising beta-caryophyllene comprises polymeric microparticles. In another embodiment, the pharmaceutical composition comprising a beta-caryophyllene comprises nanoparticles. In another embodiment, the pharmaceutical composition comprising beta-caryophyllene comprises liposomes. In another embodiment, the pharmaceutical composition comprising beta-caryophyllene comprises lipid emulsion. In another embodiment, the pharmaceutical composition comprising beta-caryophyllene comprises microspheres. In another embodiment, the pharmaceutical composition comprising beta-caryophyllene comprises lipid nanoparticles. In another embodiment, the pharmaceutical composition comprising beta-caryophyllene comprises lipid nanoparticles comprising amphiphilic lipids. In another embodiment, the pharmaceutical composition comprising beta-caryophyllene comprises lipid nanoparticles comprising a drug, a lipid matrix and a surfactant. In another embodiment, the lipid matrix has a monoglyceride content which is at least 50% w/w.

In one embodiment, compositions of the present invention are presented in a pack or dispenser device, such as an FDA approved kit, which contain one or more unit dosage forms containing the active ingredient. In one embodiment, the pack, for example, comprise metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

In one embodiment, it will be appreciated that the beta-caryophyllene can be provided to the individual with additional active agents to achieve an improved therapeutic effect as compared to treatment with each agent by itself. In another embodiment, measures (e.g., dosing and selection of the complementary agent) are taken to adverse side effects which are associated with combination therapies.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to inducing remission. In another embodiment, “treating” refers to slowing the progression of a disease. The terms “reducing”, “suppressing” and “inhibiting” refer in another embodiment to lessening or decreasing. Each possibility represents a separate embodiment of the present invention.

The term “subject” refers in one embodiment to a mammal including a human in need of therapy for, or susceptible to, a condition or its sequalae. The term “subject” does not exclude an individual that is normal in all respects. In another embodiment, the term encompasses “patient” is encompassed within the term “subject”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the examples below.

The following examples are presented in order to more fully illustrate the preferred embodiments of the invention. They should in no way be construed, however, as limiting the broad scope of the invention.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes 1-111 Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876, 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference. Other general references are provided throughout this document.

### Materials and Methods:

### Plant Material

Commiphpora gileadensis cuttings were obtained from the Dead Sea Ein Gedi Botanic Garden located in Kibbutz Ein Gedi, the Dead Sea, Israel (31° 27′N, 35° 23′E) (plants originated from the Chelsea Physic Garden, 66 Royal Hospital Road, Chelsea, London, The United Kingdom). In Ein Gedi's botanical gardens, plants were grown in the field in sandy soil, drip irrigated with tap water (drinking water originating from Ein Gedi's spring water). Each plant received 10 liters of drinking water every four days. Between November and March plants were watered every 5 days.

### Plant Extracts

Ethanolic plant extract was prepared as follows: plants were dried at 40° C. for 3 days, and plant powder was suspended in tubes with ethanol 96% (EtOH-FRUTAROM) at a ratio of 200 μg/mL and was incubated overnight at room temperature (25° C.) Following incubations, tubes were centrifuged at 13,000 revolutions per minute (rpm). The upper fluid (the extract) was removed to another tube and kept at −20° C. for further analyses.

### Chemical Compounds

β-Caryophyllene: (−)-trans-Caryophyllene, syn. β-Caryophyllene, trans-(1R,9S)-8-Methylene-4,11,11-trimethylbicyclo[7.2.0]undec-4-ene (C₁₅H₂₄), was purchased from Sigma-Aldrich, Inc. 204.35 g/mol, ≧98.5% pure, Catalog Number 22075.

Citral: syn. 3,7-Dimethyl-2,6-octadienal (C₁₀H₁₆O), 40:60% of geranial and neral mixture was used in this research as a positive control (Ref Rivki, 2005) Purchased from Sigma-Aldrich, Inc (Fluka). 152.24 g/mol, ≧95.% pure, catalog number 27450.

Staurosporine (STS): syn. Antibiotic AM-2282 (C₂₈H₂₆N₄O₃), from Sterptomyces sp. was used in this research as a positive control. Purchased from Sigma-Aldrich, Inc. 466.53 g/mol, ≧95% pure, catalog number S4400.

### Cell Culture

The following cell lines were used in this study: (1) BS-24-1: mouse lymphoma cell; (2) MoFir: Epstein-Barr virus transformed human B lymphocytes generated in the laboratory by transformation of human B cell from an anonymous donor (see below); (3) FB: normal human skin fibroblasts.

BS-24-1, and MoFir were grown in Roswell Park Memorial Institute medium (RPMI—Biological Industries Beit Haemek) supplemented with 2 mM L⁻¹ glutamine (Biological Industries Beit Haemek), 10% fetal bovine serum (FBS—Biological Industries Beit Haemek), 100 U/mL penicillin and 100 mg/mL streptomycin (Biological Industries Beit Haemek). FB were grown in Dulbecco/Vogt modified Eagle's minimal essential medium (DMEM—Biological Industries Beit Haemek) supplemented with 2 mM L-1 glutamine, 20% FBS, 100 U/mL penicillin and 100 mg/mL streptomycin. All the cells were maintained at 37° C. in a water-saturated atmosphere of 5% CO₂.

### EBV Transformation

To establish the MoFir cells, B cells from whole blood were prepared by Ficoll -Hypaque density gradient centrifugation. The cells were infected with the B95-8 strain of Epstein-Barr virus. RPMI medium (Biological Industries Beit Haemek) was used for cell culture. The EBV B lymphocyte cell line was maintained in RPMI supplemented with 10% FBS, 100 U/mL penicillin and 100 mg/mL streptomycin. The culture was maintained at 37° C. in 5% CO₂ atmosphere. The medium was changed on a twice per weekly basis.

### Separation and Identification of Volatiles Components in Gas Chromatography-Mass Spectrometry (GC-MS)

Solid phase micro-extraction (SPME) samples and 1 μl from the extracted samples were analyzed in a computerized GC-MS (GC-6890N) equipped with a Mass Selective (MS)-5973 Network (Electron ionization 70 eV) detector of Agilent Technologies (CA, USA). A capillary column, Rtx-5Sil MS (Restek Corporation, State College, Pa.) (30 m×0.25 mm) i.d×0.25 μm silica, was installed into the GC-MS. The carrier gas, helium (He) was in mode of constant flow of 1 mL/min. The extraction samples were introduced into the column in a ‘Splitless’ mode, while the oil and SPME samples were introduced in a ‘split’ mode ratio of 1:50. Temperatures were set as followed: the injector's temperature 250° C., and both the transfer line and detector's temperature were 280° C. The column's temperature gradient was set for 50° C. for 1 minute, addition of 5° C. per minute up to 260° C., and 260° C. for ten minutes.

Component recognition was based on comparison of retention time index (RI) of the components to commercial standards and by comparison of the samples' mass spectrum with GC-MS libraries: Adams 2001, NIST-98, and QuadLib 1607.

### Essential Oil Preparation

Essential oils of Commiphora gileadensis (L.) (Burseraceae) were prepared as described in Dudai N. et al., (2000) (Dudai N, Larkov O, Putievsky E, Lerner H R, Ravid U, Lewinshon E et al. Biotransformation of constituents of essential oils by germinating wheat seeds. Phytochemistry 2000; 55: 375-82, incorporated herein by reference in its entirety).

### Results Presentation

The results presented in this study are from a minimum of five experiments with each compound. Samples were analyzed in triplicates.

### Cell Death

Cell death was assessed by incubation of cells with tetrazolium salt XTT. The production of formazan can be monitored using an ELISA reader at a wavelength of 450 nm. Cytotoxicity in compound-treated culture was expressed as follows: % survival=100×(absorbance of compound-treated cell/absorbance of ethanol-treated cells).

Apoptosis was induced by incubation of the cell lines with the compounds in their normal serum-supplemented growth medium.

Caspase-3 cellular activity assay was carried out according to the manufacturer's instructions. For measuring specific inhibition by Ac-DEVD-CHO, cell extracts were pre-incubated with the inhibitor (0.05 μM) for 10 minutes before the addition of the substrate. For assaying in vitro caspase-3 activation, BS-24-1 and MoFir cells (1.0×10⁶/mL) were incubated with β-Caryophyllene for 2 h at 37° C. Cellular extracts were prepared and then caspase-3 activity was measured.

DNA ladder analysis was performed as previously described Ofir R. et al., (1999) (Ofir R, Zhang L-, Adams J M. Interference with gene expression induces rapid apoptosisin p53-null T lymphoma cells. Cell Death and Differentiation 1999; 6: 1216-1221).

### Animal Studies

Studies evaluating the effect of C. gileadensis extract and β-caryophyllene in an in vivo animal model are performed using the humanized mouse animal model described in Sato et al., 2011 (“A novel animal model of Epstein-Barr virus-associated hemophagocytic lymphohistiocytosis in humanized mice” Blood, 2011 May 26; 117(21):5663-73, incorporated herein by reference in its entirety) or using the rabbit model described in Takashima, 2008 (“A new animal model for primary and persistent Epstein-Barr virus infection: human EBV-infected rabbit characteristics determined using sequential imaging and pathological analysis” J Med. Virol. 2008 March; 80(3):455-66, incorporated herein by reference in its entirety). C. gileadensis extract and β-caryophyllene are supplied in balanced salt solution containing 12% Tween 80 (pH 7.4) and diluted immediately before use in appropriate concentrations. Doses of 5.0, 10 or 20 mg/kg are administered for 4 consecutive days to control and EBV groups (eight animals/group) by i.p. injection of 0.1 ml per mouse or within the food or drinking water. Both C. gileadensis extract and β-caryophyllene are found to prevent EBV-induced death, viremia, leukocytosis, IFN-γ cytokinenemia, normocytic anemia, and thrombocytopenia, and increase survival time

### Example 1

### Inhibition of Cancer Cell Proliferation by a C. Gileadensis Extract

In order to investigate whether C. gikadensis extracts had anti-proliferative effects against tumor cell lines, an MTT (3-(4,5-Dimethylthiazol-2-yl)-2,5-diphenyltetrazolium bromide) assay was used. The effect of this extract was assessed in mouse (BS-24-1) and human (MoFir) cell lines using ethanol (EtOH)-based stem extracts. The concentration of cells was 1.0×10⁶/mL.

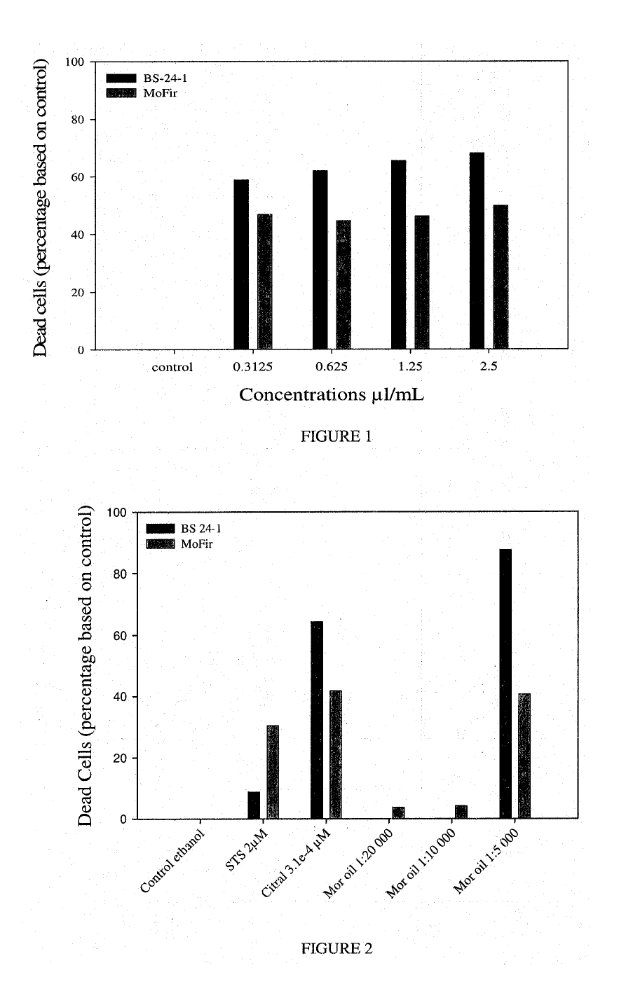

Following a 24 h incubation of cells with extracts, up to 70% of BS-24-1 cells and 50% of MoFir cells at 0.5 μl/well (IC₅₀=0.3125 and 2.5 μl/mL respectively) were dead (FIG. 1). Based on these results, the effect C. gileadensis essential oil on the tumor cell lines comparing it to two reference compounds staurosporine (STS) and citral was further assessed (FIG. 2).

Essential oil was diluted 1:5000 (stock solution:1 μl:5 μl in ethanol). 1 μl from stock solution was added into 1000 μl cell growth medium, incubated with tumor cell lines for 2 h. 87% of BS-24-1 cells and 40% of MoFir cells were killed. These results suggested that one or more components in the essential oil contributed to the tumor cell lines killing effect. Next, the essential oil volatile components were separated and identified using GC-MS. The compound list received from fractionation (Table 1) was composed mainly of terpens.

TABLE 1
Composition of the essential oil from C. gileadensis leaves
and fruits collected in Ein Gedi's botanical garden. General
chemical profile and the content percentage of
individual components are presented

| Components | % content in oil |
|---|---|
| α-Thujene | 0.64 |
| α-Pinene | 7.21 |
| Camphene | 0.18 |
| Sabinene | 21.11 |
| β-Pinene | 0.90 |
| α-Terpinene | — |
| Para-Cymene | 0.16 |
| Limonene | 0.27 |
| β-Phellandrene | 0.80 |
| (Z)-β-Ocimene | 0.34 |
| (E)-β-Ocimene | — |
| γ-Terpinene | 2.75 |
| cis-Sabinene hydrate | 0.16 |
| Terpinolene | — |
| Trans-Sabinene hydrate | 0.20 |
| allo-Ocimene | 0.28 |
| Borneol | — |
| Terpinen-4-ol | 1.26 |
| α-Terpineol | 0.33 |
| n-Decanal | — |
| Bornyl acetate | 1.66 |
| Bicycloelemene | 2.21 |
| α-Ylangene | 0.63 |
| β-Cubebene | 0.10 |
| β-Elemene | 0.53 |
| (E)-Jasmone | 0.17 |
| β-caryophyllene | 20.12 |
| β-Copaene | 1.85 |
| 6,9-Guaiadiene | 0.43 |
| Sesquiterpene hydrocarbone | 0.77 |
| Sesquiterpene hydrocarbone | 0.69 |
| Sesquiterpene hydrocarbone | 1.29 |
| α-Humulene | 0.59 |
| Dauca-5,8-diene | 0.43 |
| Germacrene D | 19.62 |
| Bicyclogermacrene | 2.91 |
| Sesquiterpene hydro carbone | 1.02 |
| δ-Amorphene | 1.35 |
| γ-Cadinene | 2.65 |
| δ-Cadinene | 0.37 |
| Nerolidol | 0.31 |
| Germacrene D-4-ol | 0.55 |

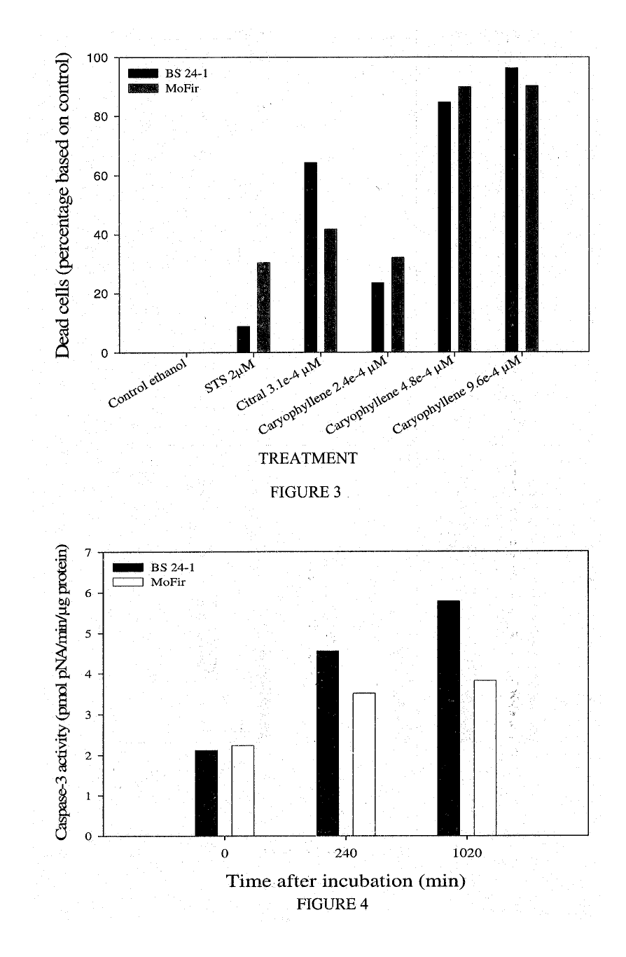

As can be seen in Table 1, beta-caryophyllene is the dominant constituent in the extract. Thus, the effect of beta-caryophyllene on tumor cell was assessed. FIG. 3 shows the unexpected impact of beta-caryophyllene after incubation for 2 h using STS and citral as references. Beta-caryophyllene induced 85-90% cell killing in both cell lines at concentrations of 4.8e⁻⁴ μM. This result strongly suggests that beta-caryophyllene is the active compound.

### Example 2

### Beta-Caryophyllene Induces Apoptosis Via Activation of Caspase-3

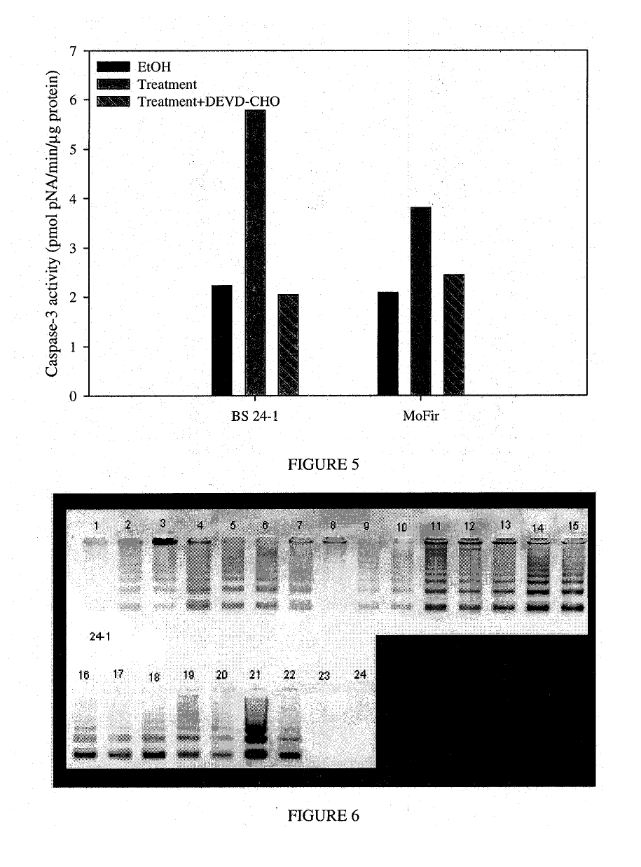

Incubation of human (MoFir) and mouse (BS-24-1) tumor cell lines for 4 h with beta-caryophyllene at low concentrations (2.4e⁻⁴ μM), resulted in activation of the enzymatic activity of caspase-3 (FIG. 4). Pretreatment of MoFir and BS-24-1 cells with the specific caspase-3 inhibitor Ac-DEVD-CHO, blocked the beta-caryophyllene induced increase of caspase-3 activity, indicating that the active enzyme in the assay in both cell lines is indeed caspase-3 (FIG. 5). The property of beta-caryophyllene as inducer of caspase-3 enzymatic activity was compared to the reference compounds STS and citral and was found to be stronger than both (data not shown).

### Example 3

### C. Gileadensis Stem Extracts and Beta-Caryophyllene Induces DNA Fragmentation-Apoptosis in Cancer Cells but not in Normal Cells (Non-Cancerous or Non-EBV Transformed Cells)

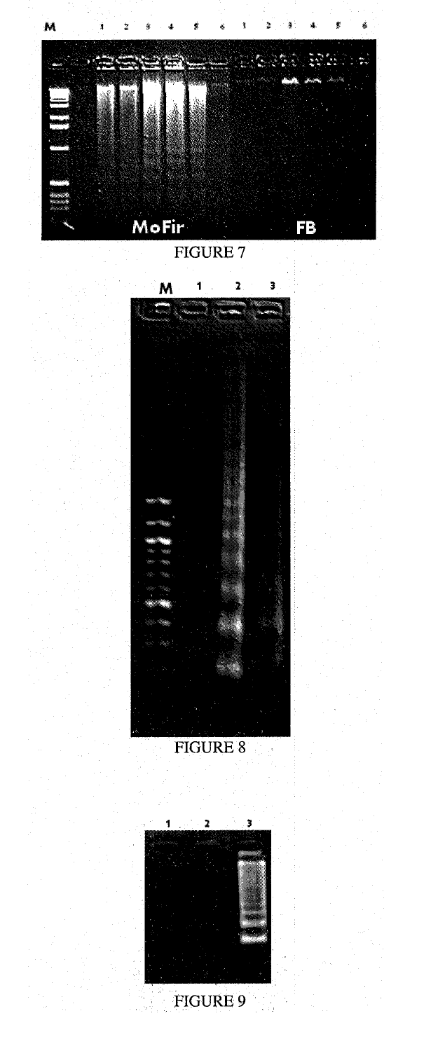

A biochemical hallmark of apoptosis is the activation of endonucleases leading to the fragmentation of the genomic DNA, which produces a characteristic ladder on agarose gel electrophoresis. When C. gileadensis stem extracts were incubated with BS-24-1 for 24 h, a DNA ladder was observed (FIG. 6). 24 h incubation of C. gileadensis stem extracts with normal cells (FB) and MoFir produced a DNA ladder only in MoFir cells. This suggests that the non-lymphoma cells an less sensitive to the apoptosis inducing effects of the treatments described herein (FIG. 7). 24 h incubation of essential oil with BS-24-1 formed a similar pattern (FIG. 8) which was repeated following a 2-h treatment of BS-24-1 cells with beta caryophyllene (FIG. 9).

Commiphora gileadensis stem extracts and essential oil showed anti-proliferative pro-apoptotic effect (exhibited via DNA ‘ladder’ and caspase-3 activation) in tumor cell lines while there was no apoptosis induction in normal cell lines (FB) (FIG. 8).

The results presented herein, unexpectedly, provide that Commiphora gileadensis extracts and beta-caryophyllene induce restrictive, differential apoptosis in diseased cells but not in normal cells (FB). The diseased cells were eliminated by apoptosis through caspase 3 activation and exhibited DNA ‘ladder’.

From the results presented herein, it can be deduced that beta-caryophyllene is at least one of the compounds responsible for C. gileadensis essential oil anti-cancerous properties. Since beta-caryophyllene is a natural product used by humans on a daily basis, it can be readily acceptable as a dietary supplement.

### Example 4

### B-Caryophyllene Inhibits the Survival of EBV-Producing Cells

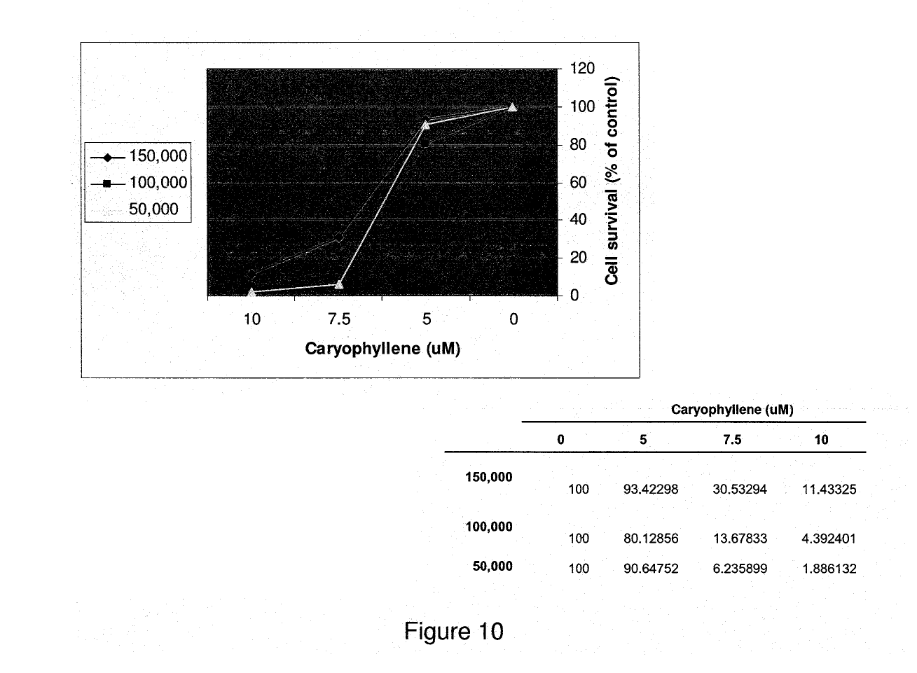

Epstein-Barr virus strain B95-8 can immortalize human B lymphocytes. B95-8 is a cell line that produces Epstein-Barr virus (EBV). Increasing the concentration of β-Caryophyllene decreases the survival of EBV-producing B95-8 cells (FIG. 10). This result fully demonstrates that β-Caryophyllene inhibits the survival of EBV-producing cells.

### Example 5

### β-Caryophyllene Inhibits the Activity of Topoisomerase I

Topoisomerase I introduces a nick in the DNA backbone allowing the rotation of one DNA strand around the second DNA strand. The DNA break is extremely transient and is re-ligated almost immediately. When an inhibitor of Topoisomerase I is present, it binds to the Topoisomerase I-nicked DNA complex or to the Topoisomerase I itself and this prevents the re-ligation of the nicked strand or the initial nick and rotation, respectively. Topoisomerase I and II activities are required for Epstein-Barr Virus replication.

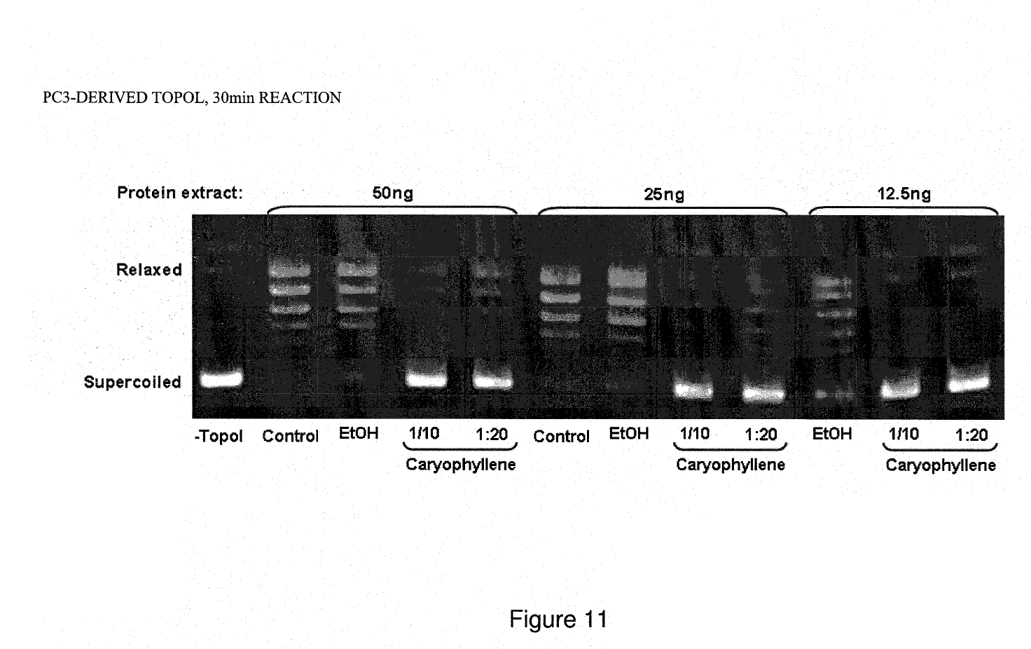

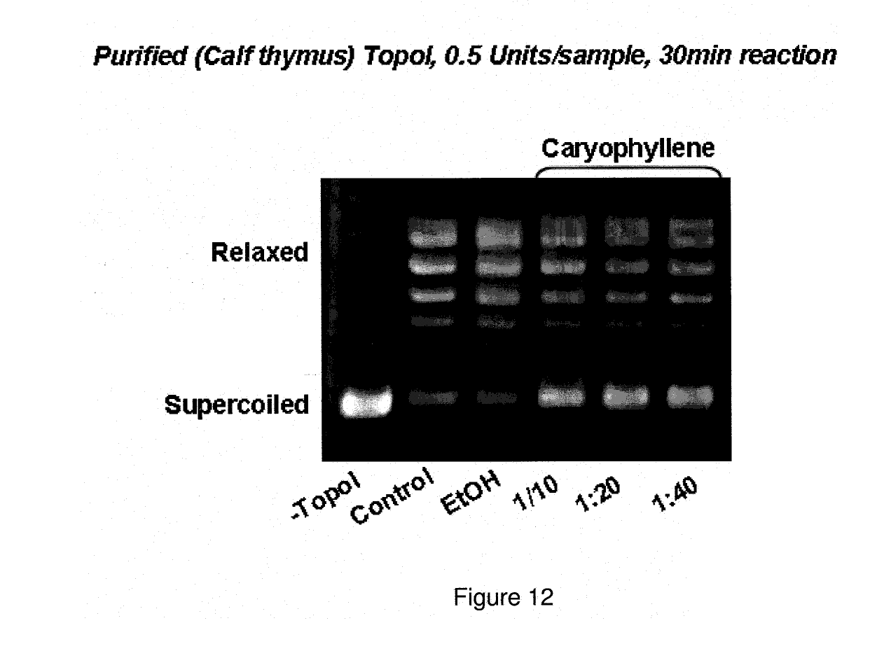

β-Caryophyllene inhibits the activity of Topoisomerase I (FIGS. 11 and 12), demonstrating that β-Caryophyllene inhibits the replication of Epstein-Barr virus.

### Example 6

### β-Caryophyllene Inhibits the Activity of Nuclear Factor Kappa B (NF-kB)

Nuclear factor kappa B (NF-kB) is an important regulator in cell fate decisions, such as programmed cell death and proliferation control, and is critical in tumorigenesis.

Human viruses use NF-kB to induce their genes. Inhibitors of NF-kB inhibit EBV transformation, disrupt gamma herpes viral latency, and CMV infectivity.

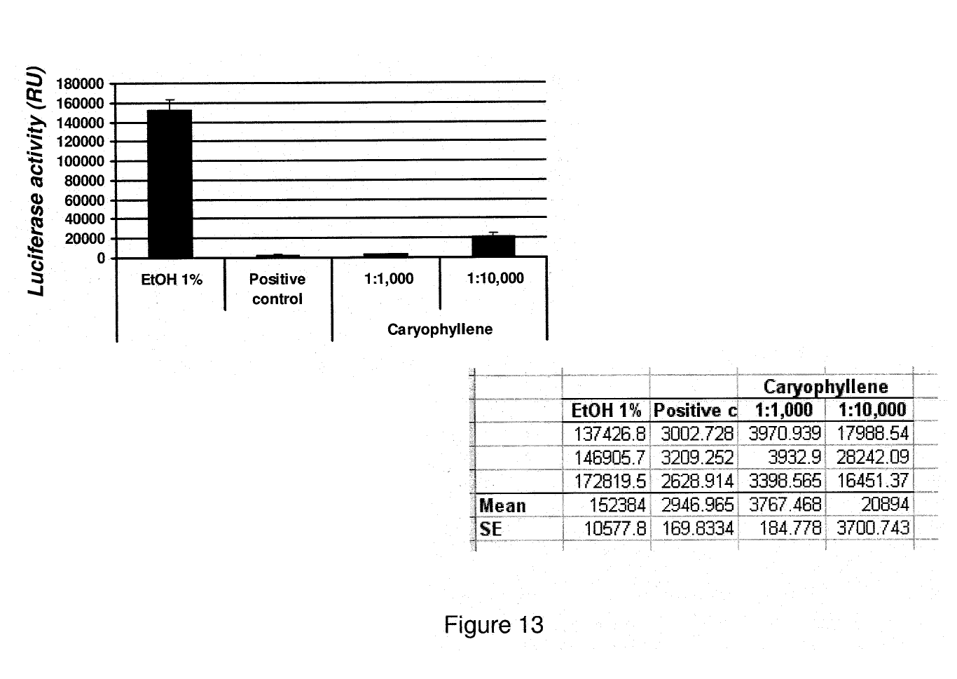

β-Caryophyllene inhibits the activity of NF-kB (FIG. 13), which is required for Epstein-Barr virus replication. Thus, FIG. 13 provides additional evidence that β-Caryophyllene inhibits the replication of Epstein-Barr virus.

Having described the embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method for inhibiting the proliferation of a cell transformed by Epstein-Barr virus (EBV), comprising the step of contacting said cell with an effective amount of a composition comprising Beta-Caryophyllene, thereby inhibiting the proliferation of said cell transformed by EBV.

2. (canceled)

3. (canceled)

4. The method of claim 1, wherein said Beta-Caryophyllene
(a) inhibits the activity of Topoisomerase I thereby inhibiting replication of said EBV in said cell; or
(b) inhibits the activity of Nuclear factor kappa B (NF-kB), thereby inhibiting replication of said EBV in said cell; or
(c) induces apoptosis in said cell;
or any combination thereof.

5. (canceled)

6. (canceled)

7. The method of claim 1, wherein said cell comprises a cancer cell, a lymphoma cancer cell, or a B cell lymphoma cancer cell.

8. (canceled)

9. (canceled)

10. The method of claim 1, wherein said composition comprises a plant extract, essential oils of a plant, a resin of a plant, or a stem or leaf extract of a plant, or any combination thereof.

11. The method of claim 10, wherein said plant is Commiphora gileadensis.

12. A method of treating a subject infected by Epstein-Barr virus (EBV) or a subject having at least one cell transformed by EBV, comprising the step of administering to said subject an effective amount of a composition comprising Beta-Caryophyllene, thereby treating said subject infected by EBV or said subject having at least one cell transformed by EBV.

13. The method of claim 12, wherein said Beta-Caryophyllene
(a) inhibits the replication of said EBV in said subject by inhibiting the activity of Topoisomerase I, Nuclear factor kappa B (NF-kB), or a combination thereof; or
(b) induces apoptosis of an EBV-transformed cell in said subject;
or any combination thereof.

14. (canceled)

15. (canceled)

16. The method of claim 1, wherein said apoptosis is differentially induced in said EBV-transformed cell compared with a non-cancer cell or a non-EBV transformed cell.

17. The method of claim 12, wherein said subject infected by EBV is afflicted with infectious mononucleosis, has a lymphoma, has a B cell lymphoma or has an EBV-associated disease, or any combination thereof.

18. (canceled)

19. (canceled)

20. The method of claim 12, wherein said treating comprises inhibiting reactivation of a latent infection of B-lymphocytes, reducing the risk of a malignancy, or a combination thereof, in said subject.

21. (canceled)

22. The method of claim 12, wherein said composition comprises a plant extract, essential oils of a plant, a resin of a plant, or a stem or leaf extract of a plant, or any combination thereof.

23. The method of claim 22, wherein said plant is Commiphora gileadensis.

24. A method for inducing apoptosis in a cancer cell, comprising the step of contacting said cell with an effective amount of a composition comprising Beta-Caryophyllene, thereby inducing apoptosis in said cancer cell.

25. The method of claim 24, wherein said inducing apoptosis in a cancer cell is differentially inducing apoptosis in a cancer cell compared with a non-cancer cell.

26. The method of claim 24, wherein said composition comprises a plant extract, essential oils of a plant, a resin of a plant, or a stem or leaf extract of a plant, or any combination thereof.

27. The method of claim 26, wherein said plant is Commiphora gileadensis.

28. The method of claim 24, wherein said cancer cell comprises a lymphoma cell or a B lymphocyte cancer cell.

29. (canceled)

30. The method of claim 24, wherein said cancer cell is infected with Epstein-Barr virus (EBV).

31. A method of treating a lymphoma or an EBV-associated disease in a subject, comprising the step of inducing apoptosis in a lymphoma cell in said subject, wherein said inducing apoptosis in a lymphoma cell in said subject comprises administering to said subject an effective amount of a composition comprising Beta-Caryophyllene, thereby treating said lymphoma or EBV-associated disease.

32. The method of claim 31, wherein said treating lymphoma or EBV-associated disease induces apoptosis in a cancer cell, differentially inducing apoptosis in said cancer cell compared with a non-cancer cell.

33. The method of claim 31, wherein said composition comprises a plant extract, essential oils of a plant, a resin of a plant, or a stem or leaf extract of a plant, or any combination thereof.

34. The method of claim 33, wherein said plant is Commiphora gileadensis.

35. A method of treating a lymphoma or an EBV-associated disease in a subject, comprising the step of administering to said subject an effective amount of a composition comprising a stem-cell extract, essential oils of a plant, a resin exudate of a plant, or a stem or leaf extract of a plant, or any combination thereof, thereby treating said lymphoma or EBV-associated disease.

36. The method of claim 35, wherein said plant is Commiphora gileadensis.
